(19) United States
(12) Patent Application Publication
(10) Pub. No.: US 2006/0275920 A1
(43) Pub. Date: Dec. 7, 2006

(54) Apparatus and method for discriminating among lateral flow assay test indicators

(76) Inventors: John Francis Petrilla, Palo Alto, CA (US); Annette C. Grot, Cupertino, CA (US)

Correspondence Address: AVAGO TECHNOLOGIES, LTD., P.O. BOX 1920, DENVER, CO, 80201-1920, US

(21) Appl. No.: 11/140,978

(22) Filed: Jun. 1, 2005

(51) Int. Cl. G01N 33/558; G01J 3/42

(52) U.S. Cl. 436/514; 356/319

(57) ABSTRACT

An apparatus for analyzing a lateral flow test strip having a plurality of types of markers for binding to analytes corresponding, respectively, to the types of markers. The apparatus includes a plurality of emitters corresponding, respectively, to the plurality types of markers on the lateral flow test strip. Each emitter emits light in a predetermined range near an optimum absorption wavelength for the corresponding type of marker to excite the marker.

### BACKGROUND OF THE INVENTION

### DESCRIPTION OF THE RELATED ART

Lateral flow assays are commonly used diagnostic tools. For example, lateral flow assays are commonly used in home pregnancy tests and to test blood sugar levels. Some assays, such as a home pregnancy test, rely on a user's observation of a change in a test strip. Other assays, such as those used to test blood sugar levels, provide improved readability and accuracy by using integrated optical detection to analyze a lateral flow test strip.

For example, FIG. 1 is a diagram illustrating a conventional apparatus used to analyze a lateral flow test strip. Referring now to FIG. 1, a sample is placed on a conventional lateral flow test strip 10. Via capillary action, the sample laterally flows across the test strip to a detection zone 18, which is shown in FIG. 1. As the sample laterally flows across the test strip to detection zone 18, analyte 15, which is the portion of the sample to be detected, may be bound to some type of marker. Thus, the concentration of markers present in detection zone 18 is related to the concentration of analyte.

Once the analyte 15 has reached the detection zone of the test strip 10, a broad spectrum light source 20 is typically used to excite the bound marker, causing the bound marker to emit an optical signal 25. This optical signal 25 is typically read by a detector 30, which thereby detects the presence, absence or concentration of analyte 15. Conventionally, the detector 30 is a photodiode which produces an electrical output corresponding to the intensity of the detected signal. Detector 30 is connected to an external display device (not illustrated) to display, for example, a numerical readout or other indication corresponding to the electrical output of detector 30.

Further, optical component 40, which may be an optical lens, filter, or lens/filter combination, may be provided to improve the performance of the reader.

With some lateral flow assays, ambient light may be sufficient to excite the marker bound to the analyte 15. If so, the assay reader might not include a light source. Additionally, if excitation of the marker produces, for example, a color change visible to the naked eye, the assay reader might not include a detector.

Many conventional lateral flow assay readers are reusable, and are used in conjunction with a disposable lateral flow test strip.

### BRIEF DESCRIPTION OF THE DRAWINGS

These and other aspects and advantages of the invention will become apparent and more readily appreciated from the following description of the preferred embodiments, taken in conjunction with the accompanying drawings of which:

FIG. 1 (Prior Art) is a diagram illustrating a conventional apparatus used to analyze a lateral flow test strip.

FIG. 2 illustrates a lateral flow assay reader according to an embodiment of the present invention.

FIGS. 3 and 4 illustrate a lateral flow assay reader according to additional embodiments of the present invention.

FIG. 5 is a block diagram of a lateral flow assay reader according to an additional embodiment of the present invention.

FIG. 6(a) illustrates relative absorption and fluorescence wavelengths for a marker on a lateral flow test strip.

FIG. 6(b) illustrates relative absorption and fluorescence wavelengths for a plurality of markers on a single lateral flow test strip.

FIG. 6(c) illustrates relative absorption and fluorescence wavelengths for a plurality of markers on a single lateral flow test strip, and the corresponding relative emission wavelength ranges required for excitation of the markers.

FIGS. 7 is a flowchart illustrating a method of discerning among markers, according to an embodiment of the present invention.

FIG. 8 illustrates a pulse train emitted by an emitter according to an embodiment of the present invention.

FIG. 9 is a flowchart illustrating a method of discerning among markers, according to an embodiment of the present invention.

FIG. 10 illustrates a time lag between emission by an emitter and fluorescence of a marker according to an embodiment of the present invention.

FIGS. 11(a) and 11(b) illustrate the extrapolation of peak intensity from gathered data according to an embodiment of the present invention.

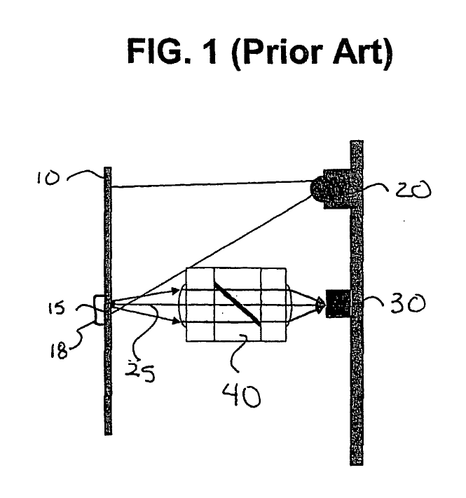

### DESCRIPTION OF THE PREFERRED EMBODIMENTS

Reference will now be made in detail to the present preferred embodiments of the present invention, examples of which are illustrated in the accompanying drawings, wherein like reference numerals refer to like elements throughout.

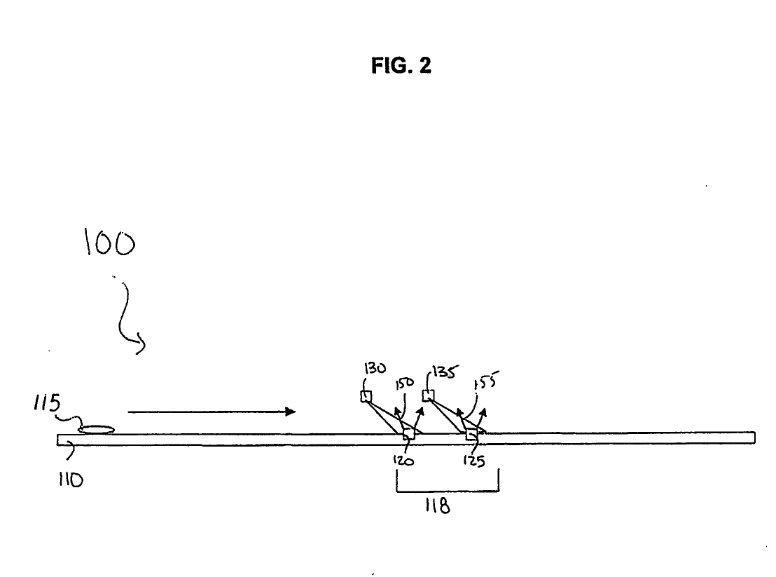

FIG. 2 is a diagram illustrating a lateral flow assay reader 100 according to an embodiment of the present invention. Referring now to FIG. 2, lateral flow assay reader 100 is used to analyze a lateral flow test strip 110 containing marker types 120 and 125. Marker types 120 and 125 each bind with a specific type of analyte contained in a sample 115, which is placed on the lateral flow test strip 110, as sample 115 is drawn across lateral flow test strip 110 via capillary action. In detection zone 118, markers 120 and 125 are shown bound to their corresponding analytes. Markers 120 and 125 are, for example, two different fluorescent markers. However, the present invention is not limited to markers 120 and 125 being fluorescent markers, and other suitable markers can be used. Further, the present invention is not limited to non-competitive assays.

Additionally, although FIG. 2 illustrates a lateral flow test strip containing two different types of markers for detecting two different analytes, the present invention is not limited to detecting only two markers. Instead, the present invention is applicable to reading a lateral flow test strip including markers for detecting a plurality of different analytes in a sample using a single lateral flow test strip.

FIG. 2 also illustrates emitters 130 and 135 corresponding, respectively, to markers 120 and 125, which are each bound to different analytes, on lateral flow test strip 110. Emitter 130 emits light in a predetermined range near an optimum absorption wavelength for marker 120. Emitter 135 emits light in a predetermined range near an optimum absorption wavelength for marker 125. Emitters 130 and 135 are, for example, light emitting diodes (LEDs). LEDs are well known. However, the present invention is not limited to emitters 130 and 135 being LEDs, and other suitable light sources can be used. Additionally, although FIG. 2 illustrates two emitters, the present invention is not limited to two emitters and may contain as many emitters as necessary to excite the number of marker types present on the lateral flow assay test strip to be analyzed, or a single broad-spectrum light source.

Excited marker types 120 and 125 then emit optical signals 150 and 155, respectively. Optical signals 150 and 155 may be detected by visual observation or, alternatively, may be detected by a detector. However, a detector is not necessary in all lateral flow assay readers.

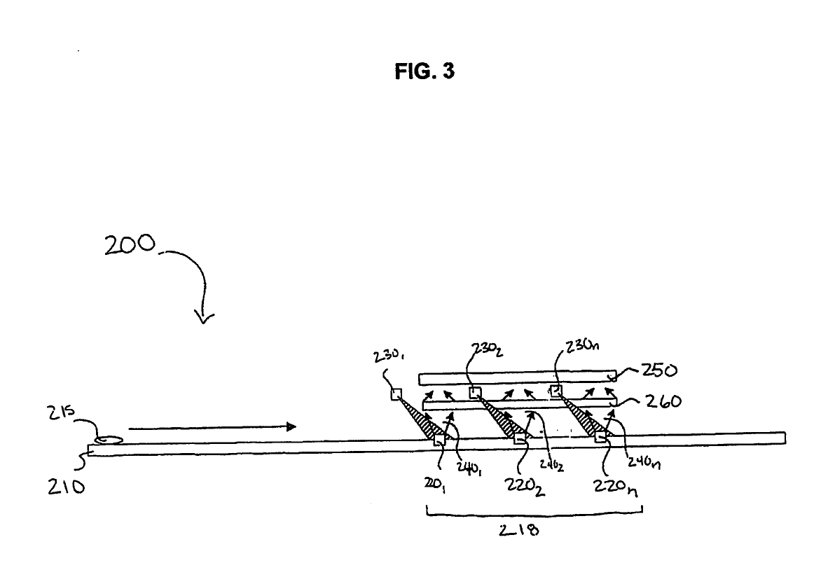

FIG. 3 is a diagram illustrating a lateral flow assay reader 200 according to an alternative embodiment of the present invention. Referring now to FIG. 3, lateral flow assay reader 200 is used to analyze a lateral flow test strip 210 containing a plurality of different types of markers 220₁, 220₂, . . . , 220ₙ. Each of the plurality of different types of markers bond to a corresponding analyte contained in sample 215, which is placed on the lateral flow test strip 210, as sample 215 is drawn across lateral flow test strip 210 via capillary action. In detection zone 218, shown, marker types 220₁, 220₂, . . . , 220ₙ are shown bound to their corresponding analytes. Marker types 220₁, 220₂, . . . , 220ₙ are, for example, different types of fluorescent markers. However, the present invention is not limited to marker types 220₁, 220₂, . . . , 220ₙ being fluorescent markers, and other suitable types of markers can be used. Further, the present invention is not limited to non-competitive assays. Additionally, the present invention is applicable to reading a lateral flow test strip including more than one type of marker for detecting different analytes in a sample using a single lateral flow test strip.

FIG. 3 also illustrates emitters 230₁, 230₂, . . . , 230ₙ corresponding, respectively, to markers 220₁, 220₂, . . . , 220ₙ, which are each bound to different analytes, on lateral flow test strip 210. Emitter 230₁ emits light in a predetermined range near an optimum absorption wavelength for marker 220₁. Emitter 230₂ emits light in a predetermined range near an optimum absorption wavelength for marker 220₂. Emitter 230ₙ emits light in a predetermined range near an optimum absorption wavelength for marker 220ₙ. These emissions excite the corresponding markers. Emitters 230₁, 230₂, . . . , 230ₙ are, for example, light emitting diodes (LEDs). LEDs are well known. However, the present invention is not limited to emitters 230₁, 230₂, . . . , 230ₙ being LEDs, and other suitable light sources can be used. Additionally, the present invention is not limited to any specific number of emitters and may contain as many emitters as necessary to excite the number of types of markers present on the lateral flow assay test strip to be analyzed.

Excited marker types 220₁, 220₂, . . . , 220ₙ then emit optical signals 240₁, 240₂, . . . , 240ₙ, respectively. Optical signals 240₁, 240₂, . . . , 240ₙ may be detected by visual observation, in which case a detector is not necessary. Alternatively, optical signals 240₁, 240₂, . . . , 240ₙ , may be detected by a detector 250. Detector 250 is, for example, a photodiode. Photodiodes are well known. However, the present invention is not limited to detector 250 being a photodiode, and other suitable detectors can be used. Additionally, although FIG. 3 illustrates a single detector 250, the present invention is not limited to a single detector and may contain any number of detectors including, for example, a detector corresponding, respectively, to each type of marker.

Lateral flow assay reader 200 may also include additional components 260 located between the excited marker types 220₁, 220₂, . . . , 220ₙ and detector 250. Such additional components 260 may include a lens, light pipes or other means to guide the optical signals 240₁, 240₂, . . . , 240ₙ emitted by excited marker types 220₁, 220₂, . . . , 220ₙ, respectively, to detector 250. Additional components 260 may alternatively include a filter to attenuate optical signals outside of a wavelength range for the markers to be detected by detector 250. Additional components 260 may also include polarizers or other measurement supporting components to cooperate with detector 250. These additional components 260 are not limited to use of a single component and may be used in any combination including, for example, a lens-filter combination. Further, different components may be placed between each of the excited marker types 220₁, 220₂, . . . , 220ₙ and the detector 250. The selection of appropriate materials for such components would be within the skill of a person of ordinary skill in the art, in view of the disclosure herein.

By using a plurality of emitters 230₁, 230₂, . . . , 230ₙ, the present invention allows for multiple lateral flow assays to be conducted concurrently on a single lateral flow test strip, reducing the need to collect multiple samples so that multiple lateral flow assays can be conducted on multiple test strips. Further, such a lateral flow assay reader would reduce the amount of equipment necessary to perform a number of different lateral flow assays which require the same sample, making it more cost-effective to run a plurality of lateral flow assays. Moreover, the present invention is more efficient in that multiple assays using the same sample are conducted concurrently. Additionally, by using emitters which emit light near an optimum absorption wavelength for the corresponding type of marker, each marker type will be maximally excited for detection by the user of the device or by a detector.

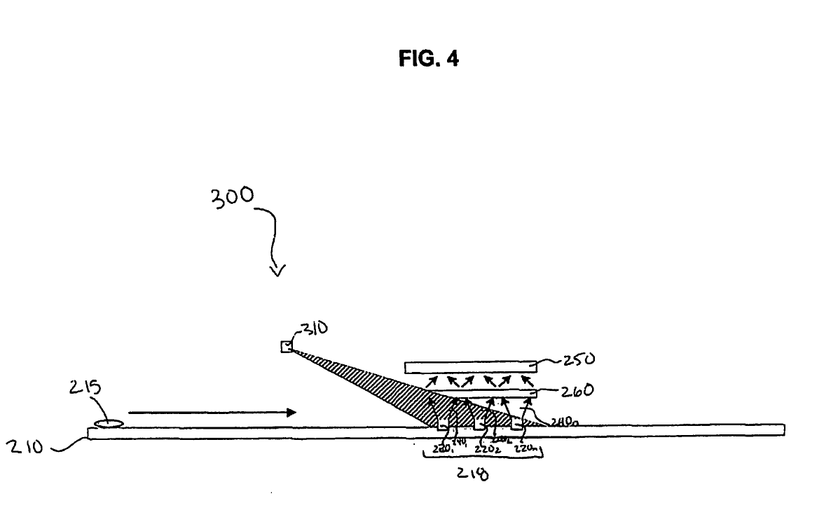

FIG. 4 is a diagram illustrating a lateral flow assay reader 300 according to an alternative embodiment of the present invention. Referring now to FIG. 3, lateral flow assay reader 300 is used to analyze a lateral flow test strip 210 containing a plurality of different types of markers 220₁, 220₂, . . . , 220ₙ. Each of the plurality of different types of markers bond to a corresponding analyte contained in sample 215, which is placed on the lateral flow test strip 210, as sample 215 is drawn across lateral flow test strip 210 via capillary action. In detection zone 218, shown, marker types 220₁, 220₂, . . . , 220ₙ are shown bound to their corresponding analytes. Marker types 220₁, 220₂, . . . , 220ₙ are, for example, different fluorescent markers. However, the present invention is not limited to marker types 220₁, 220₂, . . . , 220ₙ being fluorescent markers, and other suitable types of markers can be used. Further, the present invention is not limited to non-competitive assays. Additionally, the present invention is applicable to reading a lateral flow test strip including more than one marker for detecting different analytes in a sample using a single lateral flow test strip.

FIG. 4 differs from FIG. 3 in that it illustrates a single tunable emitter 310, rather than a plurality of emitters. The emitter 310 is tunable to emit light in a predetermined range near an optimum absorption wavelength for each of the types of markers 220₁, 220₂, . . . , 220ₙ located on lateral flow test strip 210. The tunable emitter may be, for example, a tunable light source.

As in FIG. 3, excited marker types 220₁, 220₂,. . . , 220ₙ then emit optical signals 240₁, 240₂, . . . , 240ₙ, respectively. Optical signals 240₁, 240₂, . . . , 240ₙ may be detected by visual observation, in which case a detector is not necessary. Alternatively, optical signals 240₁, 240₂, . . . , 240ₙ may be detected by a detector 250. Detector 250 is, for example, a photodiode. Photodiodes are well known. However, the present invention is not limited to detector 250 being a photodiode, and other suitable detectors can be used. Further, the present invention is not limited to non-competitive assays. Additionally, although FIG. 4 illustrates a single detector 250, the present invention is not limited to a single detector and may contain any number of detectors including, for example, a detector corresponding, respectively, to each marker type.

Lateral flow assay reader 300 may also include additional components 260 located between the excited marker types 220₁, 220₂, . . . , 220ₙ and detector 320. Such additional components 260 may include a lens, light pipes or other means to guide the optical signals 240₁, 240₂, . . . , 240ₙ emitted by excited marker types 220₁, 220₂, . . . , 220ₙ, respectively, to detector 250. Additional components 260 may alternatively include a filter to attenuate optical signals outside of a wavelength range for the marker types to be detected by detector 250. Additional components 260 may also include polarizers or other measurement supporting components to cooperate with detector 250. These additional components 260 are not limited to use of a single component and may be used in any combination including, for example, a lens-filter combination. Further, different components may be placed between each of the excited marker types 220₁, 220₂, . . . , 220ₙ and the detector 250. The selection of appropriate materials for such components would be within the skill of a person of ordinary skill in the art, in view of the disclosure herein.

By using a tunable emitter 310, the present invention allows for multiple lateral flow assays to be conducted using a single lateral flow test strip, reducing the need to collect multiple samples so that multiple lateral flow assays can be conducted on multiple test strips. Further, such a lateral flow assay reader would reduce the amount of equipment necessary to perform a number of different lateral flow assays which require the same sample, making it more cost-effective to run a plurality of lateral flow assays. Moreover, by using a tunable emitter capable of emitting light near an optimum absorption wavelength for the corresponding type of marker, each type of marker will be maximally excited for detection by the user of the device or by a detector.

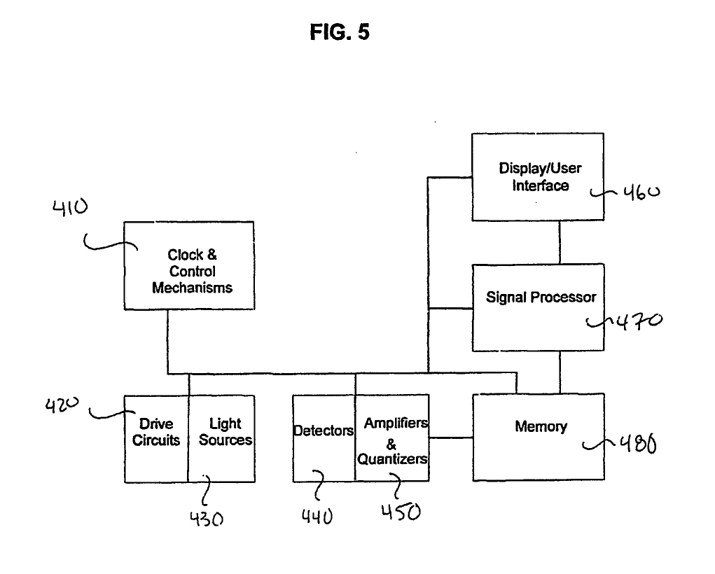

FIG. 5 is a block diagram of a lateral flow assay reader according to an additional embodiment of the present invention. The lateral flow assay readers described above, as illustrated in FIGS. 2-4, may include any combination of additional features illustrated in the block diagram of FIG. 5. First, clock and control mechanisms 410 may be included to, for example, determine the lag time between emission of light by an emitter and emission of optical signal by the corresponding excited marker. Clock and control mechanisms 410, however, are not required.

Further, drive circuit 420 controls the emission of emitter 430. Emitter 430 may be, for example, a plurality of light sources such as LEDs or a tunable light source. In a non-limiting example, drive circuit 420 may pulse emitter 430 to create a pulse train or pulse emitter 430 in a pseudo random pulse sequence. In another non-limiting example, drive circuit 420 may modulate the intensity of emitter 430. Control of emitter 430 by drive circuit 420 is not, however, limited to these embodiments.

Detector 440 is, for example, a photodiode, although other suitable detectors may be used. Detector 440 may be coupled with amplifiers and quantizers 450 to enhance signal detection.

The optical signals detected by detector 440 may be displayed on a display device 460. The optical signals detected by detector 440 may be analyzed using signal processor 470, using conventional signal processing techniques. Conventional signal processing techniques are well known in the art. Memory 480 may store, for example, information from the detector 440, information from signal processor 470, or information from user interface 460.

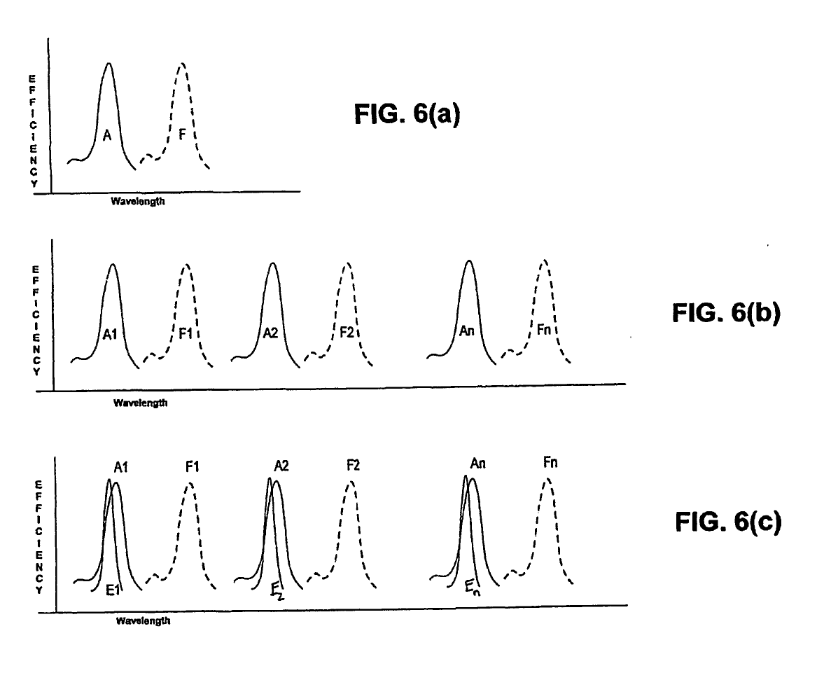

FIG. 6(a) illustrates relative absorption and fluorescence wavelengths for a marker type on a lateral flow test strip. The curve labeled A indicates absorption, and the curve labeled F indicates fluorescence.

FIG. 6(b) illustrates relative absorption and fluorescence wavelengths for a plurality of types of markers on a single lateral flow test strip. The curves labeled A₁, A₂, . . . Aₙ indicate absorption, and the curves labeled F₁, F₂, . . . , Fₙ indicate fluorescence.

FIG. 6(c) illustrates relative absorption and fluorescence wavelengths for a plurality of types of markers on a single lateral flow test strip, and the corresponding relative emission wavelength ranges required for excitation of the types of markers. The curves labeled A₁, A₂, . . . Aₙ indicate absorption, the curves labeled F₁, F₂, . . . , Fₙ indicate fluorescence, and the curves labeled E₁, E₂, . . . , Eₙ indicate emission by an emitter.

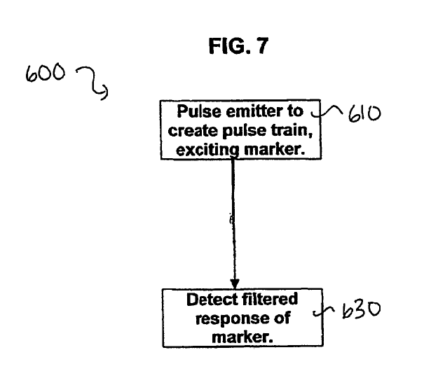

FIG. 7 is a flowchart illustrating a method 600 of discerning among types of markers, according to an embodiment of the present invention. In operation 610, an emitter is pulsed to create a pulse train. This pulse train excites a type of marker, such as a fluorescent dye. Moving to operation 630, the response of the excited type of marker is detected.

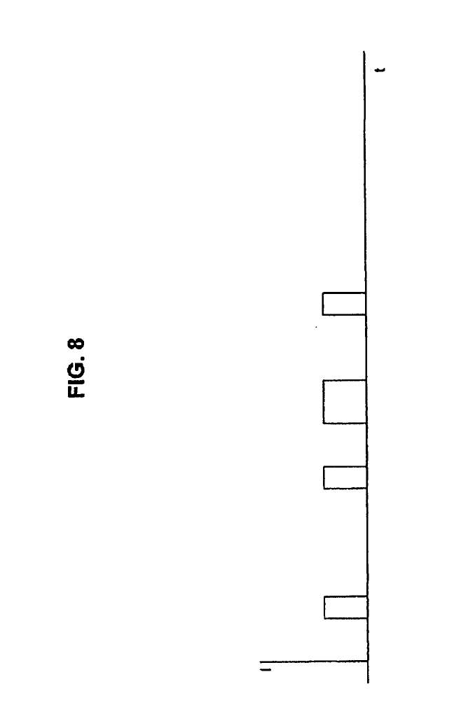

FIG. 8 illustrates an example of a pulse train emitted by an emitter according to method 600. The present invention, however, is not limited to the pulse train illustrated and may be any pulse train. Where more than one emitter is present, for example, the pulse train for each emitter would differ sufficiently from the pulse trains used by the other emitters to distinguish the emitters from one another. Pulsing the emitter or emitters allows for lower power consumption by the reader and reduces the exposure of the markers to light, as many types of markers bleach when exposed to light.

An embodiment of method 600 modulates the intensity of the pulse train emitted by the pulsed emitter.

An embodiment of method 600 pulses the emitter in a pseudo random pulse sequence.

An embodiment of method 600 filters the response of the excited marker. The response of the excited marker may be filtered before detection using, for example, an optical filter. The detected response may also be filtered, for example, based on characteristics of the detected response and pulse sequence. Filtering, however, is not limited to these types of filtering and any type of filtering may be used.

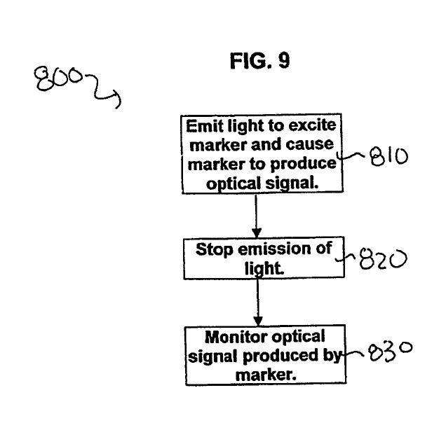

FIG. 9 is a flowchart illustrating a method 800 of discerning among types of markers, according to an embodiment of the present invention. In operation 810, an emitter emits a light to excite a type of marker on a lateral flow test strip to produce an optical signal from the excited marker type. Moving to operation 820, emission of the light is stopped. Moving to operation 830, the optical signal produced by the excited marker type after stopping emission of the light is monitored.

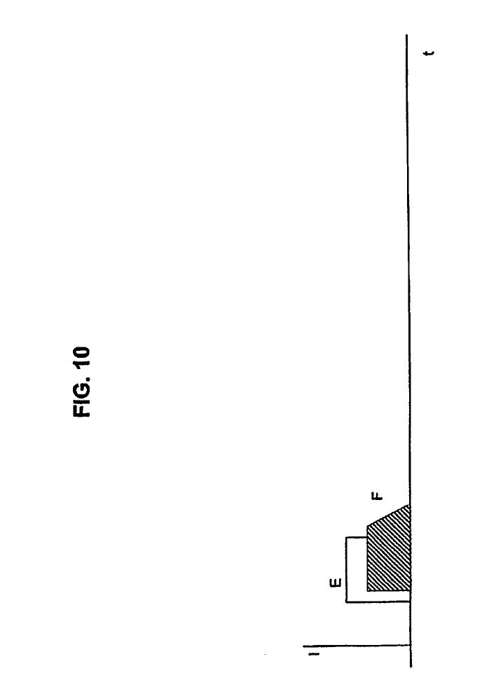

In an embodiment of method 800, monitoring the cessation of the optical signal produced by the excited marker includes measuring the time delay between stopping emission of the light and the onset of decay of the optical signal produced by the excited marker. FIG. 10 illustrates an example of a time lag between emission by an emitter and fluorescence of a marker type, where the onset of decay is measured, according to an embodiment of the present invention. This characteristic of a marker is unique to the type of marker and can be used to distinguish amongst them.

In an embodiment of method 800, stopping emission of the light causes the produced optical signal to decay and monitoring includes determining a decay rate of the produced optical signal. This characteristic of a marker is unique to the type of marker and can be used to distinguish amongst them.

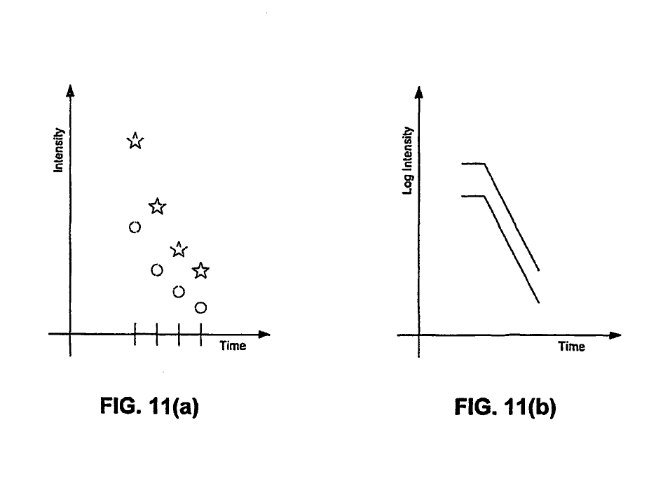

FIGS. 11(a) and 11(b) illustrate the extrapolation of peak intensity from gathered data according to the present invention. Specifically, FIG. 11(a) illustrates a plot of samples of intensity of optical signals emitted by two different excited marker types, which is measured over time. This information can be used to determine the decay rate. As the optical signals produced by the different excited marker types decay exponentially, the peak intensity of each type of marker can be determined by extrapolating back to the onset of decay the measured intensity sample information for each type of marker. As shown in FIG. 11(b), the log of the intensity can be plotted over time to determine the peak intensity. However, determination of decay rate is not limited to this method.

In an embodiment of method 800, monitoring the optical signal produced by the excited marker type after stopping emission of the light includes measuring the produced optical signal at its peak. The signal peak may also be determined by extrapolating from measurements of the produced optical signal as the produced optical signal decays.

In an embodiment of method 800, light emitted is pulsed. For example, the light may be pulsed in a pseudo random pulse sequence to excite the type of marker. However, the pulsing is not limited to a pseudo random pulse sequence and may be any pulse sequence.

In an embodiment of method 800, stopping emission of the light causes a decay of the produced optical signal and monitoring includes analyzing the trailing edge of the decay time of the produced optical signal. This trailing edge has characteristics unique to the type of marker.

The present invention allows for a plurality of lateral flow assays to be conducted using a single lateral flow test strip. Thus, the presence, absence, and relative concentration of a plurality of analytes in a single sample can be determined more expeditiously. In a clinical medical setting, for example, several lateral flow assays can be conducted on a single lateral flow test strip using a single sample of blood, rather than a separate sample for each assay. Additionally, only one lateral flow assay reader is required to analyze the lateral flow assay strip, reducing the necessary equipment and providing a lower-cost alternative.

Although it may be possible to detect the presence of a few different colored markers by observing the detection region of a lateral flow test strip, the present invention provides for discerning among more markers than would be distinguishable by the human eye. For example, closely spaced blue and yellow markers may appear as a green marker, rather than several distinct markers. Thus, rather than requiring separate, spaced detection regions for each type of marker on a test strip, the present invention allows for close spacing of multiple detection zones, or the detection of multiple types of markers located in the same detection zone. This permits smaller test strips to be used, and requires smaller sample volumes.

Additionally, by relying on unique characteristics of different types of markers, the present invention provides techniques to discern among multiple types of markers located in the same detection region.

Although a few preferred embodiments of the present invention have been shown and described, it would be appreciated by those skilled in the art that changes may be made in these embodiments without departing from the principles and spirit of the invention, the scope of which is defined in the claims and their equivalents.

What is claimed is:

1. An apparatus for analyzing a lateral flow test strip having a plurality of types of markers for binding to analytes corresponding, respectively, to the types of markers, the apparatus comprising:
a plurality of emitters corresponding, respectively, to the plurality of types of markers on the lateral flow test strip, each emitter emitting light in a predetermined range near an optimum absorption wavelength for the corresponding type of marker to excite the marker.

2. An apparatus as in claim 1, further comprising:
a detector to detect a presence, absence or concentration of an analyte in accordance with movement of an excited marker into or out of a detection zone of the lateral flow test strip.

3. An apparatus as in claim 1, wherein the markers are fluorescent dyes.

4. An apparatus as in claim 1, wherein the emitters are light emitting diodes (LEDs).

5. An apparatus as in claim 2, wherein the detector is one or more photodiodes.

6. An apparatus as in claim 2, further comprising:
an optical guide between the detector and the lateral flow test strip which guide optical signals emitted by the excited markers to the detector.

7. An apparatus as in claim 2, further comprising:
a filter between the detector and the lateral flow test strip which attenuates optical signals outside of a wavelength range for the types of markers to be detected by the detector.

8. An apparatus for analyzing a lateral flow test strip having a plurality of types of markers for binding to analytes corresponding, respectively, to the types of markers, the apparatus comprising:
a tunable optical source tunable to emit light in a predetermined range near an optimum absorption wavelength for each of the types of markers to excite the markers; and
at least one detector to detect a presence, absence or concentration of each analyte in accordance with movement of an excited marker into or out of a detection zone of the lateral flow test strip.

9. An apparatus as in claim 8, wherein the markers are fluorescent dyes.

10. An apparatus as in claim 8, wherein the detector is one or more photodiodes.

11. An apparatus as in claim 8, further comprising:
an optical guide between the detector and the lateral flow test strip which guides optical signals emitted by the excited markers to the detector.

12. An apparatus as in claim 8, further comprising:
a filter between the detector and the lateral flow test strip which attenuates optical signals outside of a wavelength range for the types of markers to be detected by the detector.

13. A method for discerning among markers, comprising:
independently pulsing an emitter to create a pulse train to excite a marker; and
detecting the response of the excited marker.

14. A method as in claim 13, further comprising modulating the intensity of the pulse train emitted.

15. A method as in claim 13, wherein the emitter is pulsed in a pseudo random pulse sequence.

16. A method as in claim 13, wherein the response of the excited marker is filtered.

17. A method comprising:
emitting a light to excite a marker on a lateral flow test strip to produce an optical signal from the excited marker;
stopping emission of the light; and
monitoring the optical signal produced by the excited marker after stopping emission of the light.

18. A method according to claim 17, wherein said monitoring comprises
measuring a time delay between stopping emission of the light and an onset of decay of the optical signal produced by the excited marker.

19. A method according to claim 17, wherein said stopping emission of the light causes a decay of the produced optical signal and said monitoring comprises
analyzing trailing edges of decay times of the produced optical signals.

20. A method according to claim 17, wherein said stopping emission of the light causes a decay of the produced optical signal and said monitoring comprises determining decay information of the produced optical signal, said decay information being used to determine peak intensity levels.

21. A method according to claim 17, wherein said monitoring comprises measuring the produced optical signal at its peak.

22. A method according to claim 17, wherein the light emitted is pulsed.
